(19) United States
(12) Patent Application Publication
(10) Pub. No.: US 2006/0079904 A1
(43) Pub. Date: Apr. 13, 2006

(54) Multirow knotless suture anchor assembly

(76) Inventor: Raymond Thal, McLean, VA (US)

Correspondence Address: Christopher J. McDonald;HOFFMAN, WASSON & GITLER, P.C., Suite 522, 2461 South Clarke Street, Arlington, VA, 22202, US

(21) Appl. No.: 11/247,208

(22) Filed: Oct. 12, 2005

Related U.S. Application Data
(60) Provisional application No. 60/617,640, filed on Oct. 13, 2004.

(51) Int. Cl. A61B 17/58

(52) U.S. Cl. 606/72

(57) ABSTRACT

A tissue repair has at least two attachment points for a suture to create a broad area of contact between tissue in bone. When two anchor points are used, the suture extends between and is attached to the two attachment points to create a suture band. When two on more attachment points are used, a first anchor point in the bone has one or more loops. The first anchor point is preferably located on a medial location at the repair site when performing rotator cuff repair. For other types of repair procedures, the first anchor point is different and located as required. The loop or loops are passed through the soft tissue at points spaced from one another. The loop or loops are then captured and secured at anchor points laterally during rotator cuff repair and spaced from the first anchor point and from one another. When complete, the loop or loops are in the form of a “V” and a large area of contact between the soft tissue and bone is established. When more than two loops are used, each loop extends from the first attachment point to another attachment point.

The application claims the benefit of provisional application 60/617,640, filed Oct. 13, 2004.

### BACKGROUND OF THE INVENTION

Arthroscopic procedures often require soft tissue to be reattached to bone. To achieve this, anchors are placed in the bone and sutures attached to the anchor are passed through the tissue to securely retain the tissue in place. The prior art has developed knotless suture anchors, such as those disclosed in U.S. Pat. Nos. 5,569,306, 5,658,313, 5,665,112, 5,709,708 and 6,045,574, the disclosures of which are incorporated herein by reference.

When making a repair of soft tissue to bone, it is advantageous to have as large an area of contact between the bone and tissue as possible. Anchor points spaced from one another in rows result in a repair having a broader area of contact. There is a need in the art for a procedure that securely attached tissue to bone over a large area of contact. The procedure must use existing components and be able to be done in a quick efficient manner with a minimum of recovery time for the patient.

It is an object of the invention to reattach tissue to bone over a large contact area.

It is another object of the invention to provide a procedure to reattach tissue to bone using a plurality of attachment points.

It is still another object of the invention to have a tissue repair avoiding arthroscopic knot tying.

It is yet another object of the invention to provide a procedure to reattach tissue to bone minimizing both the operative time and the patient's recovery time.

These and other objects of the invention will be apparent to one of ordinary skill in the art after reading the disclosure of the invention.

### SUMMARY OF THE INVENTION

A tissue repair has at least two attachment points for a suture to create a broad area of contact between tissue in bone. When two anchor points are used, the suture extends between and is attached to the two attachment points to create a suture band. When two on more attachment points are used, a first anchor point in the bone has one or more loops. The first anchor point is preferably located on a medial location at the repair site when performing rotator cuff repair. For other types of repair procedures, the first anchor point is different and located as required. The loop or loops are passed through the soft tissue at points spaced from one another. The loop or loops are then captured and secured at anchor points laterally during rotator cuff repair and spaced from the first anchor point and from one another. When complete, the loop or loops are in the form of a AV@ and a large area of contact between the soft tissue and bone is established. When more than two loops are used, each loop extends from the first attachment point to another attachment point.

### BRIEF DESCRIPTION OF THE DRAWINGS

FIG. 1 depicts a multirow repair;

FIG. 2 shows the completed repair of FIG. 1;

FIG. 3 depicts a repair with one anchor point having two loops passing through the tissue;

FIG. 4 depicts the completed repair of FIG. 3;

FIG. 5 depicts an alternative embodiment of the anchor having a single loop attached to two attachment points;

FIG. 6 depicts the first step in another type of multiple repair configuration;

FIG. 7 depicts a threaded anchor usable with the invention;

FIG. 8 depicts the anchor of FIG. 7 retaining two loops;

FIG. 9 depicts an anchor with two loops and plug capturing one loop;

FIG. 10 depicts the first step in performing a multirow repair;

FIG. 11 depicts the second step in a multirow repair;

FIG. 12 depicts the final configuration of a multirow repair;

FIG. 13 depicts the second step in a multirow repair using a plug and sleeve anchor;

FIG. 14 depicts the final configuration in the multirow repair of FIG. 13.

### DETAILED DESCRIPTION OF THE INVENTION

FIG. 1 shows tissue 10 needing to be reattached to bone 12. A first attachment device 14 retains a suture loop 16. A loop may comprise a complete circle of sutures or a strand of suture appearing as a loop when attached to the repair device. The suture passes through the tissue as seen in FIG. 1. To complete the repair, the end of the suture loop 16 is attached to a second attachment point 20 laterally spaced from the first attachment device 14. The second attachment point 20 may be a sleeve receiving a plug that retains the loop. The prior at discloses plugs that capture suture loops and are retained in sleeves but these devices have the suture attached to the same sleeve as retains the plug. In the instant procedure, the suture is attached at a point spaced from the sleeve.

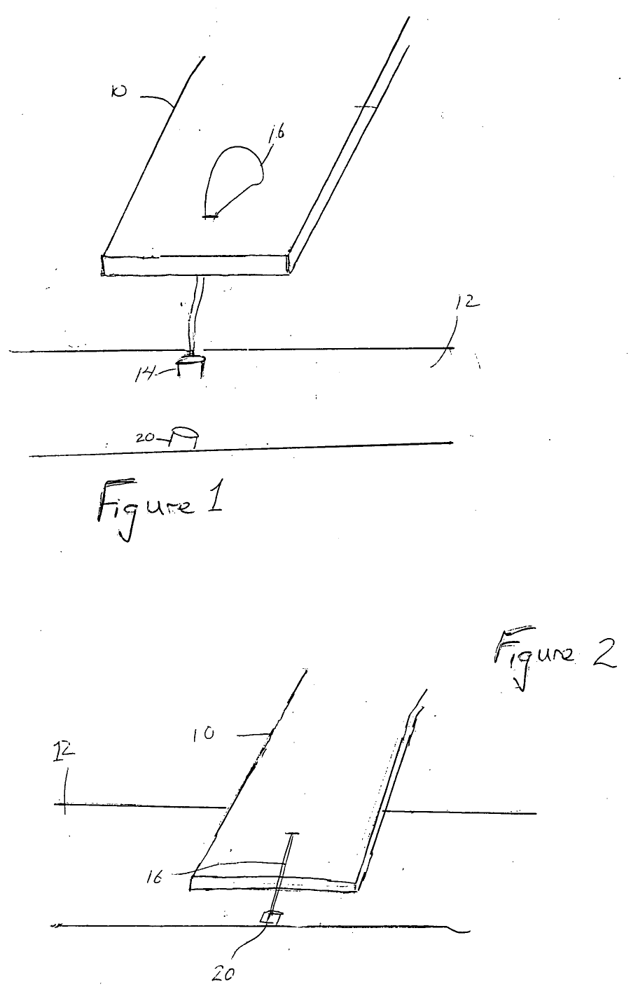

The completed repair is seen in FIG. 2 with the end of the suture loop 16 secured to the attachment point 20 to create a band between the two attachment points and over the tissue to create a broad area of contact between bone and tissue.

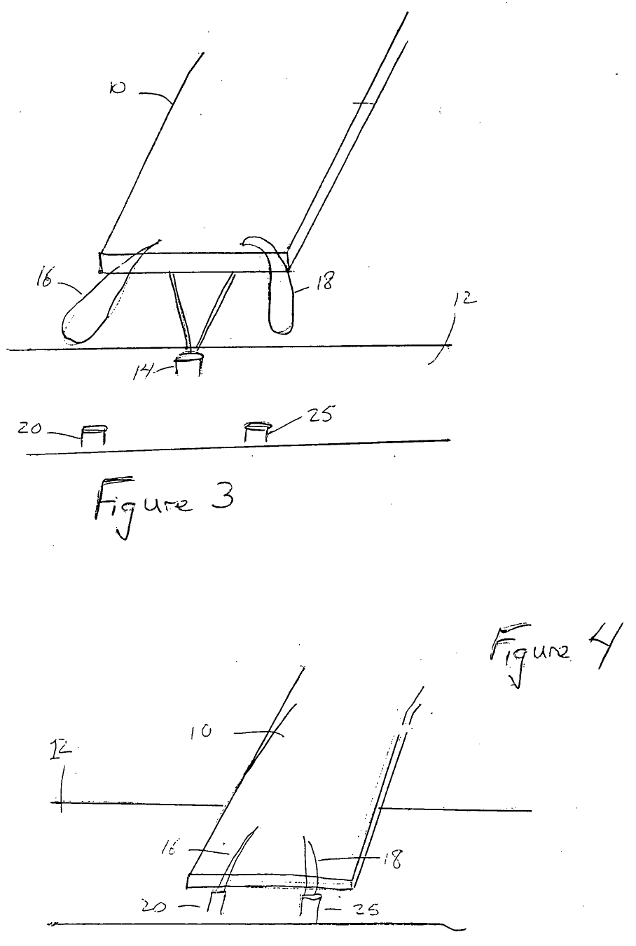

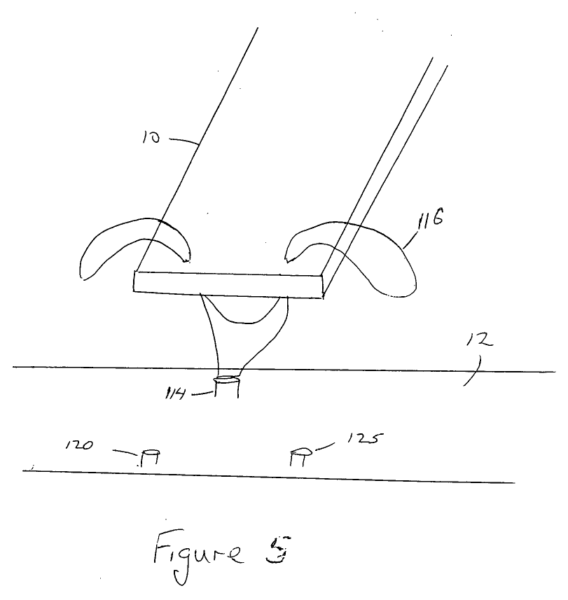

FIG. 3 shows tissue 10 needing to be reattached to bone 12. A first attachment device 14 retains a first loop 16 and a second loop 18. More than two loops may be used for a repair, as will be explained later. This strand produces an effective loop attached at any location on the repair device or molded to or with the device. Laterally spaced from the first attachment device 14 and spaced from one another are a second attachment point 20 and a third attachment point 25. As seen in FIG. 4, after the loops 16 and 18 have been attached to the second attachment point 20 and third attachment point 25, respectively, the tissue is held against the bone with the loops 16, 18 extending over the edge of the tissue. Alternatively, one or more of the loops can be repassed through the tissue one or more times prior to attachment to one of the second or third attachment points.

A third embodiment is seen in FIG. 5. The first anchor 114 has a single long suture 116 passing through the tissue at two different points. The single suture is attached to two different attachment points 120,125 to secure the tissue to the bone.

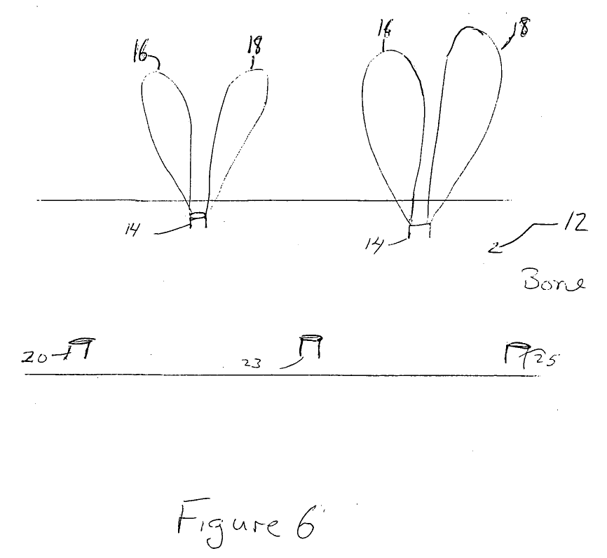

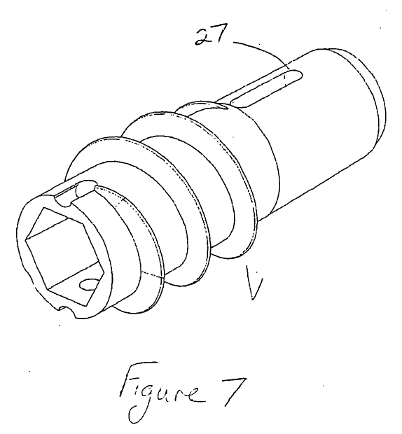

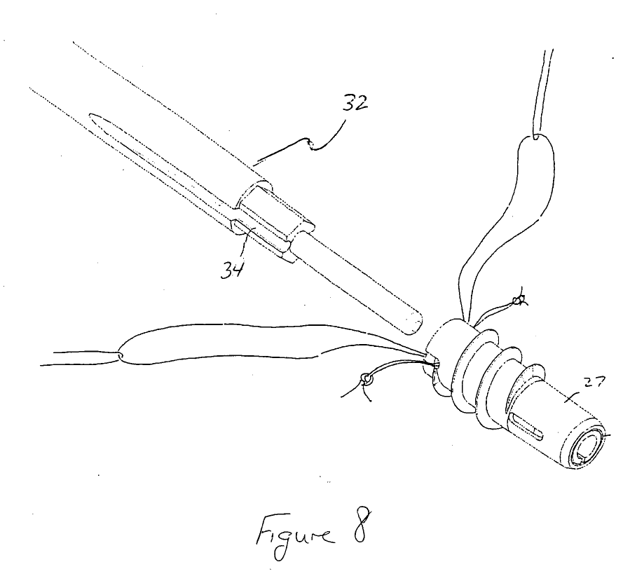

For extensive repairs along greater lengths of bone and tissue, several of the repairs comprising a first attachment device and a second and third attachment point can be used in series. One possible configuration for a multiple repair is depicted in FIG. 6. When two first attachment points 14 are used, one loop from each first attachment point may be secured into a common attachment point 23. The remaining loop from each first attachment point is then secured to a separate attachment point 20,25. In this configuration, one less anchor point is needed as the common anchor point 23 secures loops from two different first attachment points 14.

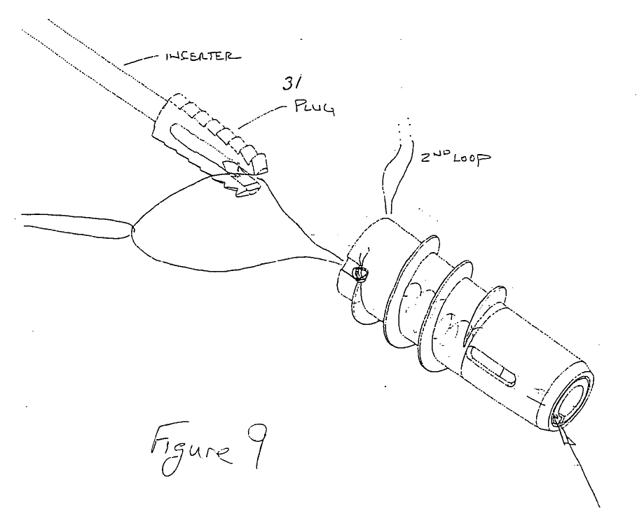

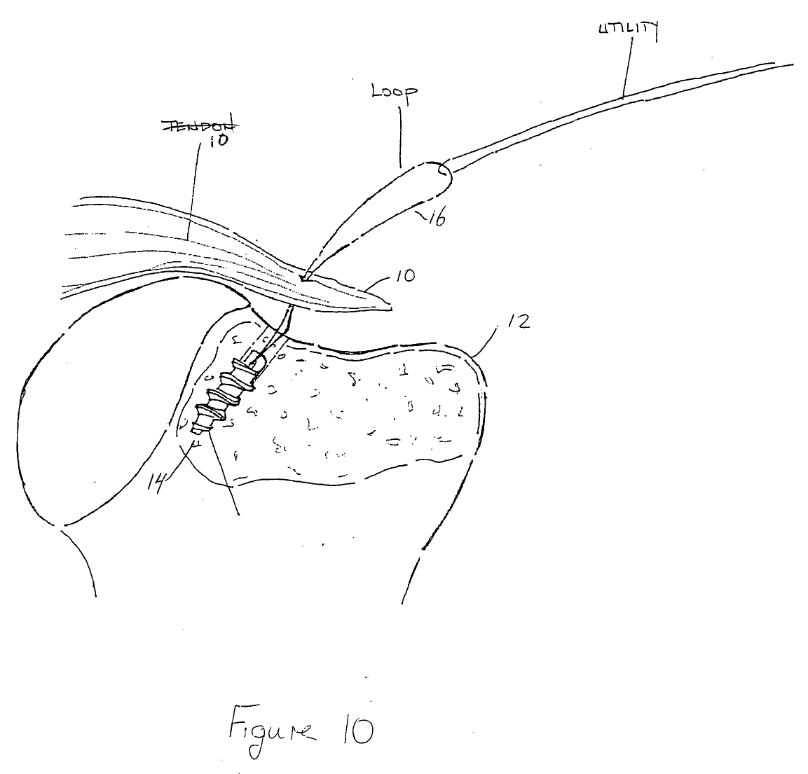

The first attachment device may be a sleeve or a screw-in anchor, each having one or more attached loops. Any type of anchor with one or more loops may be used. One possible device is shown in FIG. 7, consisting of a threaded sleeve 27 made of any suitable material such as polypropylene. FIG. 8 shows the sleeve 27 retaining two loops and a sleeve driver 32 having two suture channels. The loops are attached to the anchor by any conventional means, such as molding the two ends of a suture to the anchor. FIG. 9 depicts a plug 31 having a notch at its distal end for capturing one of the suture loops.

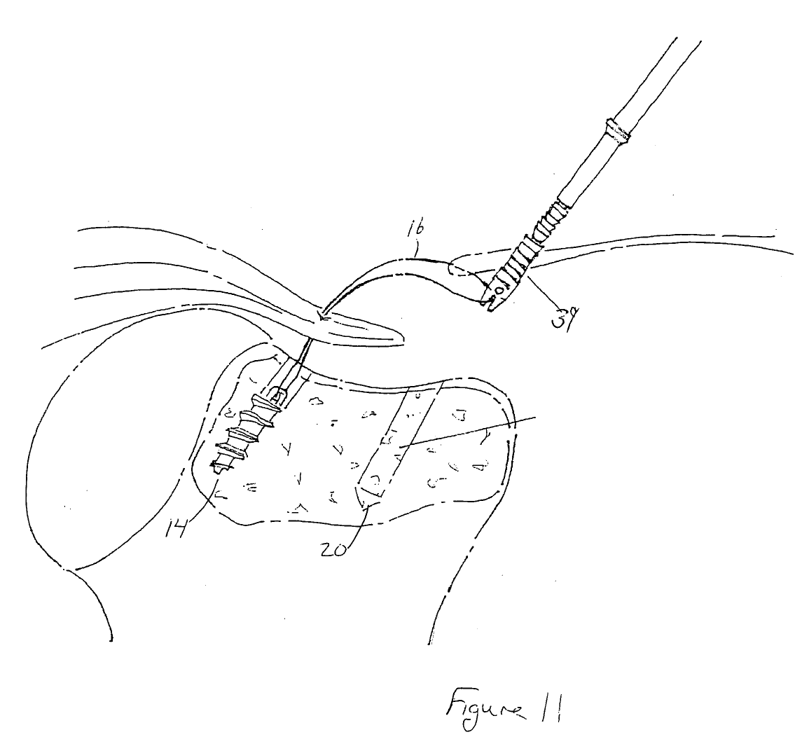

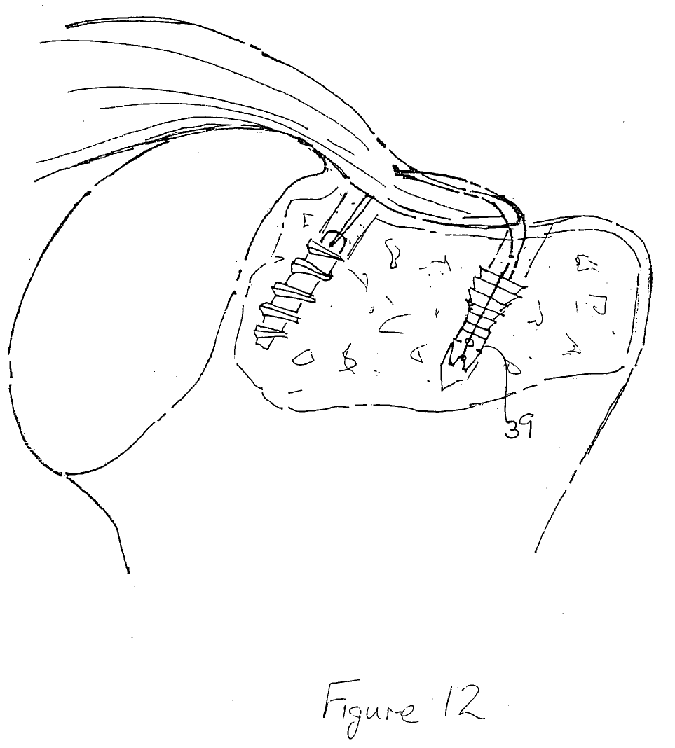

FIG. 10 depicts, in approximate cross section, the first attachment point 14. For clarity purposes only one loop 16 is shown extending through tissue 10. A second attachment point 20 is formed in the bone 12, as shown in FIG. 11. The loop is captured by an anchor 131. The anchor may be any type of anchor having bone gripping properties, such as the illustrated compression anchor. In the specific anchor shown, a slot on the end of the anchor retains the loop.

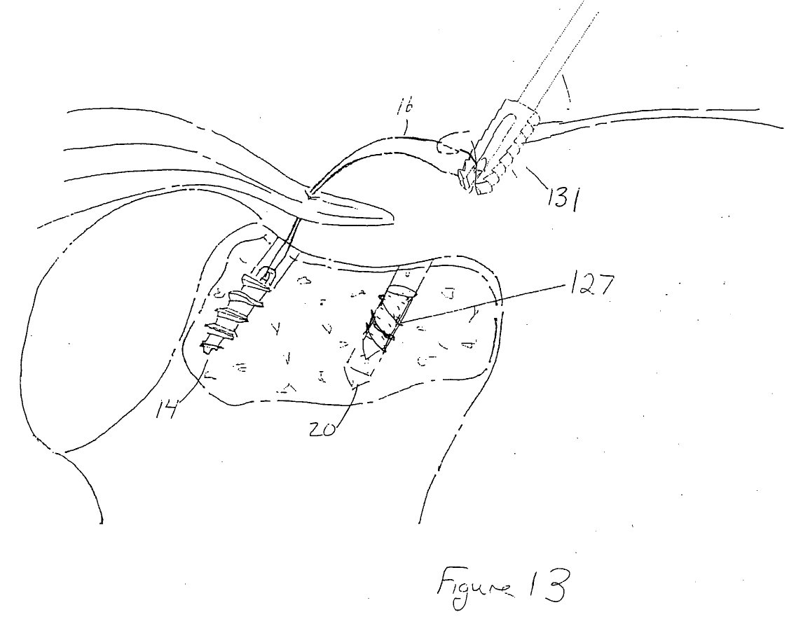

FIG. 12 shows the loop, captured by the anchor 39, retained in the second attachment point 20. The same procedure is repeated for the second loop 18 to be retained in the third attachment point 25.

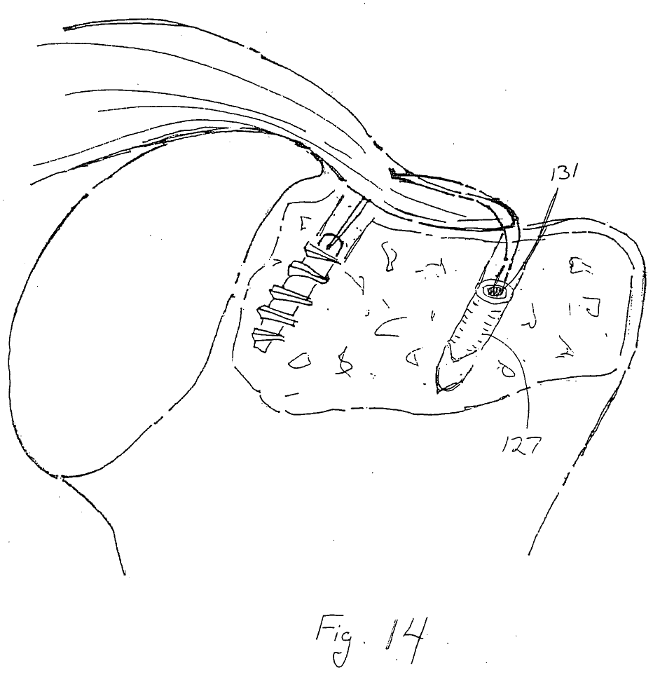

FIG. 13 is a view similar to FIG. 11 but showing the repair made with a plug and sleeve anchor. One type of sleeve anchor is disclosed in U.S. Pat. No. 6,045,574. While this patent discloses the connection between the sleeve and plug, the sleeve in the instant procedure receiving the plug does not have the suture attached to it. In this view, the sleeve 127 has been placed within the bore drilled into the bone and the plug 131 captures the loop 16.

FIG. 14 shows the plug 131 placed within the outer sleeve 127 in the attachment point 20, thereby capturing the loop 16 to create a stable, effective repair. The sleeve may be secured within the bore by any suitable means such as threads (shown), wedges or prongs. Another type of anchor enables the anchor to ratchet down inside of the sleeve to ensure the tightness of the repair.

The type of anchor used for the first attachment device 14 and in the second and third attachment points 20, 25 can be chosen from the many types of anchors in the existing art and are dictated by bone density, location of repair and surgeon preference. Regardless of the specific type of anchor, a two row repair with one or more attachment points laterally spaced from a first attachment device having one of more loops is established, resulting in a repair having greater integrity and long term stability.

I claim:

1. A tissue repair comprising
a first attachment point secured in bone, at least one loop attached to and extending from said first attachment point,
a second attachment point in the bone spaced from said first attachment point,
said at least one loop passing through tissue and secured to said second attachment point.

2. The tissue repair of claim 1, further comprising
a third attachment point spaced from said first and second attachment points,
said at least one loop passing through tissue and secured to said third attachment point.

3. The tissue repair of claim 2, wherein
said second and third attachment points comprise compression anchors.

4. The tissue repair of claim 2, wherein
said second and third attachment points comprise a sleeve and plug.

5. The tissue repair of claim 4, wherein said sleeve fits within the sleeve with a ratchet connection.

6. The tissue repair of claim 1, wherein
said at least one loop is molded to said first attachment point.

7. The method of claim 2, wherein said at least one
loop comprises a first loop and a second loop, and
said first loop is attached to said second attachment point, and
said second loop is attached to said third attachment point.

8. The tissue repair of claim 7, further comprising
a fourth attachment point secured in bone,
a third and fourth loop attached to an extending from said fourth attachment point,
a fifth attachment point secured in bone,
said third loop attached to said second attachment point, and
said fourth loop attached to said fifth attachment point.

9. A method of repairing tissue comprising
securing a first attachment in bone, said first attachment point having at least one loop,
forming a second attachment point in bone, and
attaching said at least one loop to said second attachment point.

10. The method of claim 9 further comprising
forming a third attachment point in bone, and attaching said at least one loop to said third attachment point.

11. The method of claim 10, wherein said at least one
loop comprises a first loop and a second loop and
said first loop is attached to said second attachment point, and
said second loop is attached to said third attachment point.

12. The method of claim 10, further comprising
securing a fourth attachment point in bone, said fourth attachment point having a third and fourth loop,
forming a fifth attachment point,
securing said third loop to said second attachment point, and
securing said fourth loop to said fifth attachment point.

13. A tissue repair comprising
a first attachment point secured in bone,
a suture loop attached to and extending from said first attachment point,
a second attachment point in the bone spaced from said first attachment point,
said suture loop passing through tissue and secured to said second attachment point.

14. The tissue repair of claim 13, wherein said second attachment point is a sleeve and plug, said plug capturing the suture loop and retained in said sleeve.

15. The tissue repair of claim 14, wherein said plug is retained in said sleeve by a ratchet connection.
